(19) United States
(12) Patent Application Publication
(10) Pub. No.: US 2021/0022641 A1
(43) Pub. Date: Jan. 28, 2021

(54) WEARABLE MULTI-MODAL BIO-SENSING SYSTEM

(71) Applicant: The Regents of the University of California, Oakland, CA (US)

(72) Inventors: Siddharth Siddharth, La Jolla, CA (US); Aashish Patel, Los Angeles, CA (US); Tzyy-Ping Jung, San Diego, CA (US); Terrence J. Sejnowski, La Jolla, CA (US)

(21) Appl. No.: 17/068,824

(22) Filed: Oct. 12, 2020

Related U.S. Application Data
(63) Continuation of application No. PCT/US2019/027394, filed on Apr. 12, 2019.
(60) Provisional application No. 62/656,890, filed on Apr. 12, 2018.

(51) Int. Cl. A61B 5/053; A61B 5/0476; A61B 5/16; A61B 5/00; G06N 3/08

(52) U.S. Cl. CPC A61B 5/0533; A61B 5/0476; A61B 5/163; A61B 5/0024; A61B 5/725; A61B 2560/0214; A61B 5/0006; G06N 3/08; A61B 2562/0219; A61B 2562/182; A61B 5/7285

(57) ABSTRACT

A multi-modal bio-sensing apparatus is disclosed including a first sensor module comprising a photoplethysmogram (PPG) sensor configured to produce a first output representative of a blood volume of a human user, wherein the PPG sensor is configured to remove from the first output an error signal due to movement of a user; a second sensor module comprising an electroencephalogram (EEG) sensor configured to produce a third output representative of brain neural activity of the user; a third sensor module comprising an eye-gaze camera configured to capture a gaze direction of one or more eyes of the user; and a wireless communications transceiver coupled to receive sensor data from the first sensor module, the second sensor module, or the third sensor module and configured to wirelessly transmit the received sensor data from the first sensor module, the second sensor module, or the third sensor module out of the multi-modal bio-sensing apparatus.

### CROSS REFERENCE TO RELATED APPLICATIONS

This patent document claims priority to and benefits U.S. Provisional Patent Application No. 62/656,890, entitled “WEARABLE MULTI-MODAL BIO-SENSING SYSTEM,” filed on Apr. 12, 2018. The entire content of the above patent application is incorporated by reference as part of the disclosure of this patent document.

### TECHNICAL FIELD

This patent document relates to bio-sensors and a system for capturing, recording, and analyzing bio-sensor data.

### BACKGROUND

Research in multi-modal bio-sensing has traditionally been restricted to well-controlled laboratory environments. Such bio-sensing modalities measure electroencephalogram (EEG), photoplethysmogram (PPG), pupillometry, eye-gaze and galvanic skin response (GSR) are typically bulky, require numerous connections, costly, hard to synchronize, and have low-resolution and poor sampling rates. Multi-modal bio-sensing has recently shown to be very effective in affective computing, research in autism, clinical disorders, and virtual reality among many others. None of the present bio-sensing systems support multi-modality in a wearable manner outside controlled laboratory environments with clean, research-grade measurements. New bio-sensors and systems for gathering bio-sensor data are needed.

### SUMMARY

In one aspect, multi-modal bio-sensing apparatus is disclosed. The apparatus includes a first sensor module comprising a photoplethysmogram (PPG) sensor configured to produce a first output representative of a blood volume of a user, wherein the PPG sensor is configured to remove from the first output an error signal due to movement of a user; a second sensor module comprising an electroencephalogram (EEG) sensor configured to produce a third output representative of brain neural activity of the user; a third sensor module comprising an eye-gaze camera configured to capture a gaze direction of one or more eyes of the user; and a wireless communications transceiver coupled to receive sensor data from the first sensor module, the second sensor module, or the third sensor module and configured to wirelessly transmit the received sensor data from the first sensor module, the second sensor module, or the third sensor module out of the multi-modal bio-sensing apparatus.

The following features may be included in various combinations. The error signal may be determined from a second output from an accelerometer attached to the compact multi-modal bio-sensing apparatus, and wherein the error signal is removed from the first output using an adaptive filter. The apparatus may further include one or more galvanic skin response (GSR) sensors configured to determine an impedance of the skin of the user. The apparatus may further include a worldview camera configured to capture a scene around the compact multi-modal bio-sensing apparatus. The apparatus may further include a battery power source to provide power to the first mode, the second mode, the third mode, and the wireless communications receiver, wherein the compact multi-modal bio-sensing apparatus is mobile with freedom for the user to move about. The apparatus may further include a headphone or speaker; at least one processor and at least one memory containing executable instructions to cause the data to be sent to another transceiver; and/or at least another memory configured to store the data prior to transmission. The one or more eye-gaze cameras may be infrared cameras. The EEG sensor may include a plurality of electrode sensors, each electrode sensor structured to include an electrode tip that is electrically conductive and an electrically conductive cage formed to enclose the electrode tip to form a Faraday cage to shield the electrode tip from external electromagnetic interference; and an EEG control module coupled to the electrode sensors to apply and receive electrical signals from the electrode sensors. The electrode tip may include silver and epoxy; and/or the Faraday cage is formed by an electrically conductive tape. The electrically conductive tape may include copper (Cu). The electrode sensor may include an amplifier circuit coupled to the electrode tip to provide electrical signal amplification and the amplifier circuit is enclosed by the Faraday cage. One or more objects captured on video from the worldview camera are identified in the gaze direction by computer vision, and an associated time-stamp recorded to indicate one or more event times around which sensor data is recorded. The computer vision may be trained on one or more classes of objects. Every data point of at least the PPG sensor, the EEG sensor, and the eye gaze camera is time-stamped for data synchronization. Data points of at least the PPG sensor, the EEG sensor, and the eye gaze camera may be time-stamped periodically for data synchronization

In another aspect, a multi-modal bio-sensing method is disclosed. The method includes sensing, by a PPG sensor, a blood volume of a user and generating an output representative of the blood volume; removing, from the output, an error signal due to a movement of the user; sensing, by an EEG sensor, brain neural activity of the user; determining, by an eye-gaze camera, a gaze direction of one or more eyes of the user; and transmitting, by a wireless transceiver, one or more of data representative of the blood volume with the error signal removed, data representative of brain neural activity, or the gaze direction of the user. The method may further include the following features in various combinations. The error signal may be determined from an accelerometer, and wherein the error signal is removed from the output using an adaptive filter. The method may include sensing, by one or more GSR sensors, an impedance of the skin of the user. The method may include capturing, by a worldview camera, a scene in an area around one or more of the PPG sensor, the EEG sensor, or the eye-gaze camera. The method may include powering, by a battery power source, one or more of the one or more of the PPG sensor, the EEG sensor, or the eye-gaze camera, and the wireless transceiver.

### BRIEF DESCRIPTION OF THE DRAWINGS

FIG. 1A depicts a comparison between the disclosed system to other bio-sensing systems.

FIGS. 1B depicts an example of the miniaturized PPG sensor and a scale reference size, in accordance with some example embodiments

FIG. 2 depicts an example of a block diagram for adaptive noise cancellation, in accordance with some example embodiments.

FIG. 3A depicts an example of an EEG sensor with and scale reference, in accordance with some example embodiments.

FIG. 3B shows an example of a low impedance electrode tip made of an epoxy of silver with an external copper Faraday cage, in accordance with some example embodiments.

FIG. 4 depicts examples of eye-gaze overlaid on the world view and pupil detection, stimuli detection in real-time, and EEG with real-time ICA, PPG and accelerometer signals capture, in accordance with some example embodiments.

FIG. 5 shows an example block diagram of a system including sensors, data processing, and wireless transmission, in accordance with some example embodiments.

FIG. 6. depicts example images of a system, in accordance with some example embodiments.

FIG. 7. depicts an example image of a system and user including integrated headset with world camera, EEG sensors, battery, EEG reference electrode, eye camera, earlobe PPG sensor, headphone/speaker connector, and controller, in accordance with some example embodiments.

FIG. 8 depicts examples of output voltages for 10-second waveforms from the earlobe PPG sensor before and after ANC using the vertical acceleration signal as a noise reference during walking, in accordance with some example embodiments.

FIG. 9 depicts examples of Bland-Altman plots, in accordance with some example embodiments.

FIG. 10 depicts examples of gaze accuracy and precision, in accordance with some example embodiments.

FIG. 11 depicts examples of angular precision, in accordance with some example embodiments.

FIG. 12 depicts example plots of EEG signals acquired by two sensors and a correlation, in accordance with some example embodiments.

FIG. 13 shows an example of steady-state visually evoked potentials (SSVEP) keypad used as visual stimulation, and an example of an accuracy plot, in accordance with some example embodiments.

### DETAILED DESCRIPTION

This patent document provides for a mobile system for capturing and processing real-time sensor data about a human or animal subject. Sensor data can include electroencephalogram (EEG), photoplethysmogram (PPG), pupillometry, a camera viewing the same field as the user (worldview camera), and/or user eye-gaze data. The system is battery powered and can be worn by the user untethered to any other objects or devices. Data from the various sensors is time synchronized by a common clock generated by an embedded processor included with the sensors in the system. In this way, data from the sensors can be synchronized in time to determine what objects are being viewed by the user and record the sensor outputs indicating the user's response to those objects. Computer vision techniques can be used to identify objects in time-stamped images from a camera. The identified objects with the associated time-stamp can be correlated with the other sensor data to reduce the amount of image data associated with the other sensor data. For example, the eye-gaze and worldview camera may determine that the user is looking at an object identified by the computer vision technique to be their home. Time-stamped sensor data can be associated with the time-stamped identification of their hope to produce combined data that has “home” associated with the recorded sensor data.

This patent document also discloses EEG sensor designs that can use dry electrodes without the application of a liquid and provide various features to improve the EEG measurements. For example, the disclosed EEG sensors filter the noise out right at the sensor level before transmission of the signals to the acquisition circuitry. In some implementations, the disclosed EEG sensors provide a mechanism for shielding the EEG sensors from ambient noise in the environment by the use of a Faraday cage around the EEG sensors. For example, the disclosed EEG sensors can be implemented based on a silver epoxy paste with copper to elongate the life of the sensors many folds compared to Ag/AgCl based coating used in other EEG sensor designs. The disclosed EEG sensors can be used to penetrate under human hair to have continuous contact with the scalp for improved EEG measurements.

Disclosed is a wearable multi-modal bio-sensing system capable of synchronizing, recording, and transmitting data from multiple bio-sensors -EEG, PPG, pupillometry, eye-gaze, GSR, headset, body motion, etc. while also providing task modulation features including visual stimulus tagging. Disclosed is an integrated system with multiple sensors. Moreover, the disclosed sensors are evaluated by comparing their measurements to those obtained by standard research equipment. For example, an earlobe-based motion noise canceling PPG module is evaluated against a state-of-the-art electrocardiogram (ECG) system for measuring heart rate. Dry shielded EEG sensors are evaluated by comparing the measured steady-state visually evoked potentials (SSVEP) with those obtained by research grade dry EEG sensors. An eye-gaze module is evaluated to assess its accuracy and precision. By providing a wearable platform that is capable of measuring numerous modalities in the real world and that has been benchmarked against state-of-the-art tools, the explorable questions in neural computing may be explored.

In recent years, there have been advances in the field of wearable bio-sensing. This trend has led to the development of multiple wearable bio-sensors capable of measuring GSR, PPG, etc. integrated into portable form-factors like smartwatches. The use of bio-signals for various applications such as robotics, mental health, affective computing, human-computer interaction, etc. has been expanding throughout the past decade. Using more than one bio-sensing modality is attractive because the limitations of one bio-sensor can be compensated for by using another bio-sensor. For example, EEG can be used for various non-clinical studies but lacks a robust, single application outside well-controlled laboratory environments. Since some limitations of EEG are due to the low spatial resolution, using multiple bio-sensing modalities can provide better performance than a single EEG.

Multiple sensing modalities can also be used for deep learning. Using convolution neural network (CNN) based algorithms, the need to design features has been substituted by allowing algorithms to generate models to extract relevant information from the data. Utilizing multiple modalities for CNN's is useful for extracting mutually complementary information to boost the performance. A fusion of heterogeneous modalities, such as EEG with audio and video streams is possible, instead of treating them independently. Additionally, applying CNN's to EEG and magnetic resonance imaging (MRI) provides insights into the functionality of the brain and human physiology by overcoming the low spatial resolution in the first and low temporal resolution in the latter. Multi-modal bio-sensing may be used in neurocardiology which analyzes cardiac parameters such as heart rate variability in addition to EEG for assessing emotions.

Previously, these sensing modalities are incapable of being used for research in real-world studies because they are costly, bulky, and cannot easily be integrated together for multi-modal bio-sensing. Previously, a typical strategy to attempt measurement of multiple bio-signals in the real world was to buy various sensors and then extract data from each of them separately. This, however, leads to unwieldy sensor preparation and increased post-processing synchronization effort, both of which add layers of inconvenience. No integrated headset has been proposed which can measure multiple bio-signals simultaneously in a synchronized manner. The problem of not being able to collect data in real-world environments is compounded by the lack of techniques to automatically recognize and tag various events (e.g. meaningful stimuli, objects, etc.). The standard process employed for event (or object) tagging requires an individual to manually tag the various stimuli from frame to frame in a video stream. This process is cumbersome, time-consuming, and laborious. Furthermore, the stimulus onset is not measured with fine-resolution or is ill-defined in such setups. A solution is to use eye-gaze with fixations and saccades to infer the stimulus onsets. This allows for pinpointing of the visual region, but still requires processing for tagging which may be addressed with computer vision algorithms.

Previous bio-sensors have not been compact and cost-effective as well as not providing sufficient performance in real-world applications. A multi-modal bio-sensing system such as the one disclosed herein should be capable of synchronizing multiple data streams and should be packaged in a compact form factor for easy use. Thus, for example, the use of wet electrodes for measuring electrocardiogram (ECG) or EEG which may even require placing sensors over the chest is undesirable for real-world research setups. The disclosed subject matter addresses the above limitations with bio-sensors capable of measuring physiological parameters in real-world experiments with automatic visual tagging and integrating the sensors in the form of a compact wearable headset.

Disclosed is an earlobe-based, high-resolution PPG sensor that is capable of measuring heart-rate and heart-rate variability as well as providing raw PPG data from the earlobe. Using adaptive noise cancellation and placement at the earlobe to minimize movement, the PPG sensor is also able to minimize noise due to motion. Also disclosed are dry EEG sensors capable of actively filtering the EEG signal while being able to shield them from outside electrostatic noise. These EEG sensors are used with a high-sampling ultra-low noise analog to digital converter (ADC) module. Also disclosed is a dual-camera-based eyeglass capable of measuring eye-gaze (overlaid on the wearer's or user's field of view), pupillometry, fixations, and saccades. Data acquisition from all the sensors is then performed using an embedded system, which synchronizes the various data streams. These data streams can then be saved on the embedded system or wirelessly transmitted in real-time for display. A framework such as a control framework executed in hardware automatically tags visual stimuli in real-world scenarios with the user's eye-gaze over the various bio-sensing modalities. The framework is scalable in that it can be expanded to include any other bio-sensing modalities.

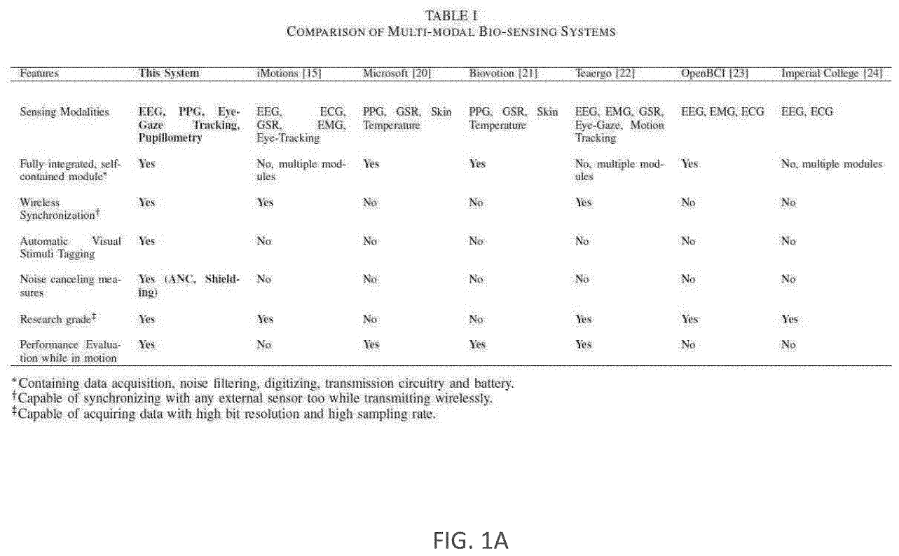

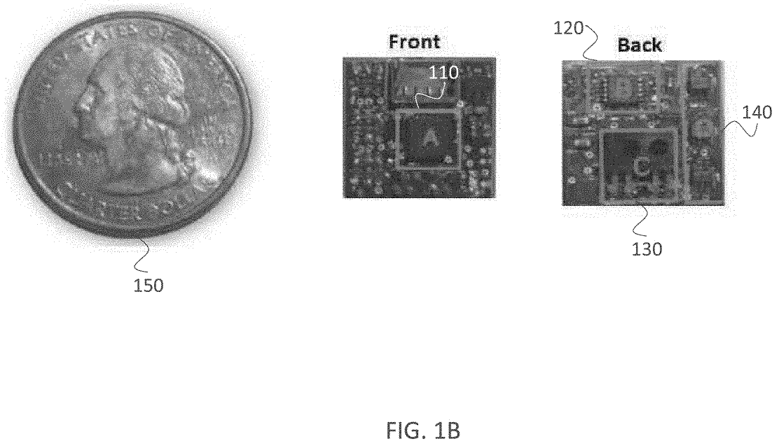

FIG. 1A at Table 1 compares the disclosed system to other state-of-the-art bio-sensing systems. Clearly, the disclosed system performs as well or better than all the existing bio-sensing systems. Detailed below are various bio-sensing modules in the disclosed system.

In real-world applications, PPG has been substituted for ECG due to the ease it offers in measuring heart-rate. It does not require using wet electrodes over the chest and can easily be integrated onto watches or armbands. But, it has its own limitations. First, most of the available PPG sensors do not have sampling rate high enough and fine ADC resolution to measure heart-rate variability (HRV) in addition to heart rate. HRV has been shown to be a good measure of emotional valence and physiological activity. Secondly, PPG sensors over the arm or wrist tend to be noisy because of the constant motion of limbs in performing real-world tasks. On the other hand, PPG systems designed for the earlobe also suffer from noise due to walking or other head and neck movements. In the rare case when noise filtering is used in PPG, the hardware design is bulky due to the size of the circuit board used in the setup. The raw PPG signals once acquired may be sent to a computer wirelessly without any timestamps or band-pass filtering to extract relevant frequency band. This tends to be noisy as the PPG signal is not amplified before transmission and poses the problem of unable to synchronize with other bio-sensing modalities.

EEG sensors come in dry or wet-electrode based configurations. The wet electrodes either require the application of gel or saline water during the experiment and hence are not ideal outside laboratory environments. Dry electrodes typically do not have a long service life since they are generally made of Ag/AgCl or gold (Au) coating over a metal, plastic or polymer, which tend to wear off. Furthermore, coating Ag/AgCl is a costly electrochemical process. It has also been shown that the use of active EEG sensors is less noisy than using passive EEG sensors. EEG sensors may need to be shielded the signals from stray electrical noise such as electrostatic noise in hostile environments.

Eye-gaze tracking systems tend to be bulky and may even require the user to place his/her face on a chin rest. Even when they are compact, these systems are not mobile and the user has to be constantly in its field of view. These limitations restrict their use outside laboratories where illumination varies and the user is mobile at all times. Furthermore, such systems only work in measuring eye-gaze as being pointed over a display monitor and not in real world. They are unable to overlay the gaze over the wearer's view if the display screen is not in his/her field of view. The solution may be to use headset mounted eye-gaze systems, but they tend to use a laptop instead of a small embedded system for processing and viewing the camera streams. Thus, the laptop has to be carried in a bag, restricting the wearer's freedom of movement.

To tag the stimulus with various bio-sensing modalities, previously the norm has been to use a key/button press, or fixing the onset and order of stimuli on a display, or timing it with a particular event etc. But, in real-world scenarios such methods either cannot be used due to the mobile nature of the setup or induce a sense of uncertainty which has to be removed by manual tagging. Such manual tagging is laborious and time-consuming. A viable solution is to tag stimuli automatically after recognizing them in the wearer's field of view. However, this may lack information about whether the user was actually focusing on the stimuli or rather was looking at some other point in his/her field of view. Using the camera capturing the wearer's field of view face action units classification can be used to capture and record the emotions of other people in his/her view. Wearer is used interchangeably with user herein.

Previous experimental setups were wired and did not have compactness to form a headset/smartwatch etc. but rather tended to just attach various sensors placed on the user, which were connected to one or more data acquisition systems. This further reduced the mobility for experiments outside laboratories. The use of independent clocks for each of the different modality can further complicate synchronizing the various modalities. The timestamps from each clock have to be arranged and synchronized which can be done after acquiring the data and not in real-time. For real-time display, transmitting data streams from sensors over Wi-Fi or Bluetooth may introduce varying latency. Thus, a solution is a closely packed hardware system that synchronizes the various data streams while acquiring them in a wired manner and using only one clock (that of the embedded system itself). The synchronized streams can then be either recorded or sent to a display screen that does not affect either the compact nature of hardware or synchronization in software framework.

An earlobe-based PPG sensor module is disclosed below. The PPG sensor module is compact (e.g., 1.6 cm×1.6 cm×0.6 cm) and sandwiched to the earlobe using two small neodymium magnets (see, FIG. 1B). The PPG sensor module houses an infrared (IR) emitter-detector for measuring PPG (e.g., Vishay TCRT 1000), a 3-axis accelerometer (e.g., Analog Devices ADXL 335), a high- precision (16-bit) and high-sampling rate (e.g., 100 Hz.) ADC (e.g., Texas Instruments ADS 1115), and a third-order analog high- gain band-pass filter (e.g., BPF, cutoff 0.8-4 Hz using three Microchip MCP6001 op-amps).

FIG. 1B depicts example images of the miniaturized PPG sensor and a scale reference size. FIG. 1B at 110 depicts a 3-axis accelerometer, at 120 a 100 Hz 12-bit ADC, at 130 an IR emitter and receiver, and at 140 a third-order filter bank. The PPG sensor may be worn behind a wearer's ear.

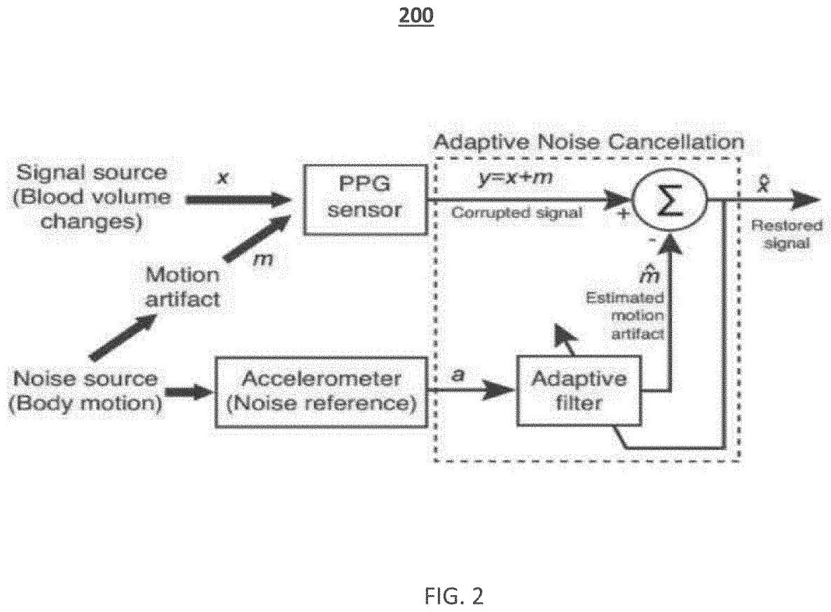

The PPG signal can be amplified using a high-gain amplifier and a band-pass filter with a predetermined frequency band is extracted. The filtered PPG data along with the accelerometer data can be digitized using an analog-to-digital (ADC) converter. The digitized data may then be transmitted via a wireless transceiver. In this way, the PPG sensor module filters the signal and digitizes the signal for transmission and/or determination of heart rate and heart rate variability that may be transmitted in addition to the digitized signal or instead of the digitized signal. The on-board accelerometer can be used for at least two purposes; first, to measure and monitor head movements because the sensor is fixed on the earlobe with reference to the position of the user's face; second, the accelerometer provides a measure of noise due to motion which can be removed from the PPG signal using an adaptive noise-cancellation (ANC) filter (see, FIG. 2). FIG. 2. depicts an example block diagram of an ANC configuration, in accordance with some example embodiments. The filter can be implemented inside the system either on an embedded controller or on a personal computer that receives the raw data. The filter may be used to generate a model of noise due to motion (such as while walking) from the reading of the accelerometer and reconstructing the noise-removed PPG signal. The adaptive filter shown in FIG. 2 can also be used to remove motion-induced errors from EEG and/or ECG data.

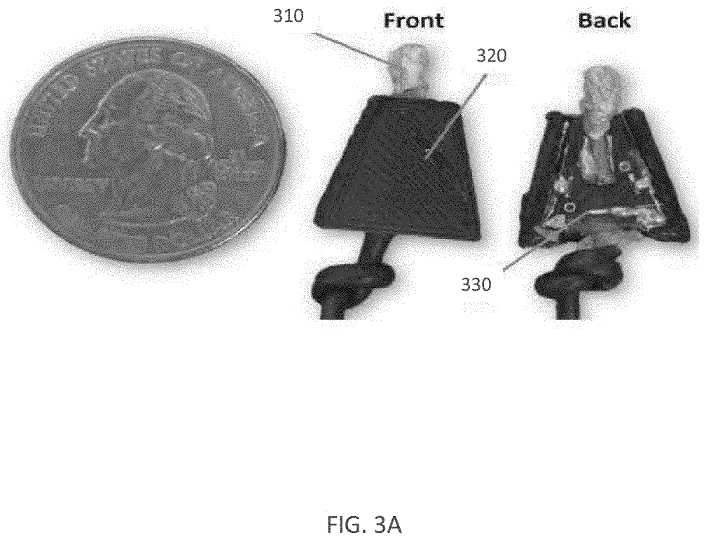

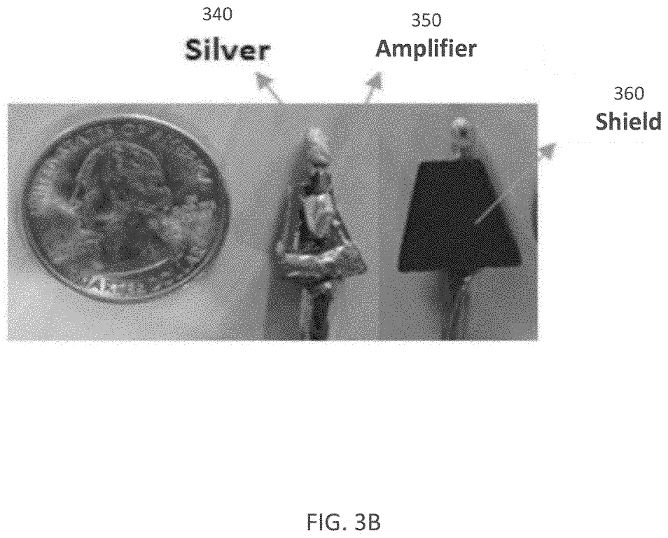

FIG. 3A depicts an example of an EEG sensor with a scale reference. At 310 is a silver (Ag) based conductive element, at 320 is a 3D printed case housing a conductive element for shielding, and at 330 is the amplifier circuitry on the PCB.

Disclosed herein are dry EEG sensors (see, FIG. 3A) which can be adjusted under the hair to measure EEG signals from the scalp. The dry EEG sensors include a conductive element made from silver (Ag) epoxy (with electrical resistivity, for example, of 0.007 Ωcm). This silver-epoxy-based conductive element enables a long-life sensor since the silver does not wear off as fast as it does on EEG sensors coated with Ag/AgCl.

The sensor may include an operational amplifier (opamp) (e.g., Texas Instruments TLV 2211) on-board to increase the EEG signal amplitude and thereby increasing the signal-to-noise ratio (SNR) of the EEG signal. In some example embodiments, the opamp may be configured in a voltage-follower configuration. Furthermore, the sensor may be enclosed in a copper (Cu) housing to shield the sensor from electromagnetic interference. The copper housing may act as a Faraday cage around the sensor. In some example embodiments, the housing may be made using conductive tape such as copper tape. The shielding prevents noise from the environment from interfering with the desired EEG signal before the signal is amplified. A band-pass filter may be included before and/or after the amplifier to reduce unwanted frequencies.

FIG. 3B shows an example of an EEG sensor including low impedance electrode tip 340, amplifier 350, and electromagnetic noise shield 360. Electrode tip 340 can be made from an epoxy material that includes silver. The silver provides high conductivity and the epoxy binds it strongly to the shield extending the service life of the electrode tip. The shield may surround the interior of the sensor and may be made from copper or another conductive metal or material. The shield may function as a Faraday cage. The assembly of the silver electrode tip is such that it can penetrate under hair easily before the start of recording. The disclosed are very compact in size. Amplifier 350 is embedded in the sensor.

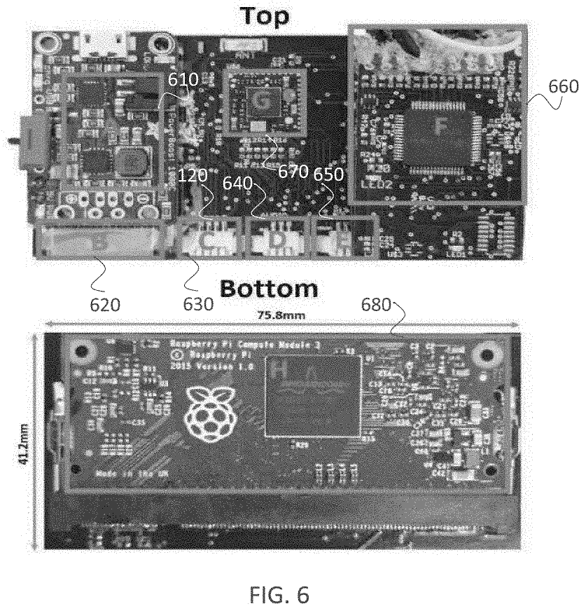

For converting the analog noise-removed EEG signal to a digital format, the disclosed system includes a 24-bit resolution high-sampling rate (up to 16 k samples/second), ultra-low input referred noise (1 μV), ADC (e.g., Texas Instruments ADS 1299). Many other resolutions, sample rates, and devices can also be used. In some example embodiments, a low-pass filter may be used before the signal is passed to the ADC. Parameters such as sampling rate, bias calculation, internal source current amplitude for impedance measurement etc. can be controlled by executable code running on a processor. In some example embodiments, the assembly can support eight (or more) EEG channels (see, FIG. 6F) whereas the design of the board is such that multiple boards can be sandwiched on top of one another to accommodate more EEG channels. For example, two such boards can be used in an example headset and hence support 16 EEG channels (Fp1, Fp2, F7, F3, Fz, F4, F8, C3, Cz, C4, P3, Pz, P4, 01, Oz, and 02 according to the International 10-20 EEG placement). Continuous impedance monitoring can be performed for each electrode in real-time to assess the quality of the EEG signal and electrode placement. Independent component analysis (ICA) can be used to separate various independent components of the signal thus separating noise due to blink, eye movement, EMG, etc.

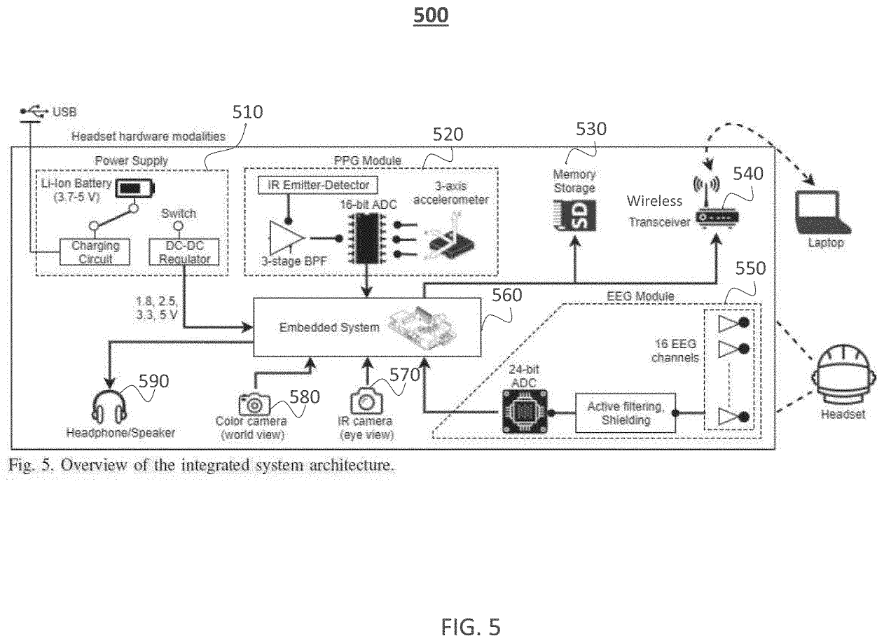

Two cameras (see, FIG. 5) can be used to assess the wearer's eye-gaze location and pupillometry parameters such as the diameter of the pupil, fixations, saccades etc. The eye cameras can include two infrared (IR) LED's (e.g., 970 nm wavelength), which are used to illuminate the region around each eye. Because the LEDs are IR, the eye camera can detect the wearer's pupil under a wide variety of illumination conditions. Pupil-detection and eye-gaze calibration code running on a processor can be used to detect the pupil and calibrate the user's gaze. A display screen (for example a laptop) may be used for the initial eye-gaze calibration step, which is done using a manual selection of natural features in the field of view. The gaze can then be superimposed on the user's view from the world camera. Both cameras can stream at 30 fps while the resolution can be adjusted as per the need of study.

A deep-learning algorithm can be used to tag various stimuli in the feed from world camera in real-time. For example, the deep-learning algorithm You Only Look Once (YOLO) can be used. The algorithm can be trained for object classes using large image databases with multiple classes. Whenever the wearer's gaze falls inside the bounding box of one of the object classes (stimuli), the bio-sensing modalities can be tagged. Hence, instead of manually tagging the stimulus during the experiment, the system can tag the information about which objects in the environment were present and where the user's gaze was fixed at various times. For example, if the wearer is looking at a person's face, his/her EEG can be time-synchronized to the gaze and analyzed to determine the level of arousal. Due to the processing requirements for using YOLO using a graphics processing unit (GPU), the stimulus tagging may be performed in real-time on a processor other than the GPU, or stored for post-processing the data.

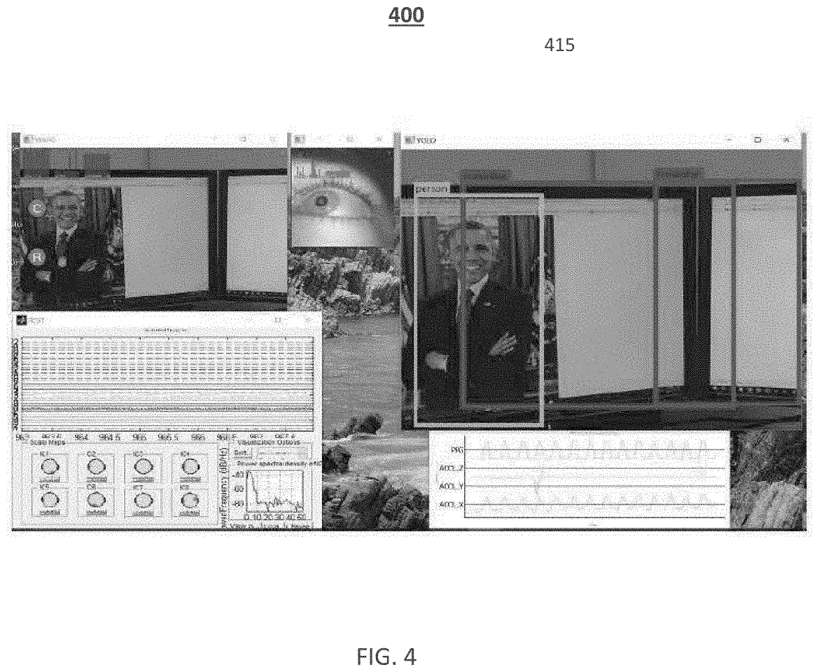

FIG. 4 depicts an example of an eye-gaze overlaid on the world view and pupil detection (top-left). Stimuli detection in real-time (top-right). EEG with real-time ICA, PPG and accelerometer signals capture (bottom panels).

The above modalities may be wired to a custom electronics board shown in FIG. 6. The board can attach to a world camera, eye camera, PPG module, and EEG module, and so on. The board includes a headphone jack which can be used for audio recording during experiments. The clock on the embedded system is common to all the modalities to enable synchronization of the data from the independent data streams. A lab streaming layer (LSL) may be used. Video streams may be compressed and transmitted wirelessly. For example, MJPEG may be used for compression, or another video compression algorithm to reduce the needed bandwidth.

FIG. 5 shows an example system diagram 500 including the sensors, data processing, and wireless transceiver. Shown are PPG module 520, EEG module 550, worldview camera 580, eve-gaze camera 570, and speakers 590. Sensors 520, 550, 670, 580, and 590, and wireless transceiver 540 are connected to a controller (or embedded system) 560. System 500 also includes memory 530 for storing sensor data. Wireless transceiver 540 may be, for example, Wi-Fi (e.g., Realtek RTL 8723BS module). Other wireless interfaces for transmission may be used as well such as WiMAX or any other wireless interface with sufficient bandwidth. The system may be powered by a lithium-ion battery 510 (e.g., Panasonic NCR18650B 3400 mAh), which may last for hours. The system can also be powered by any SVDC mobile power bank for more longer durations of continuous use.

FIG. 6. depicts an example of a system. At 610 is an example of power circuitry, at 620 is an example of a world camera connector, at 630 is an example of a PPG connector, at 640 is an example of an audio jack connector, at 650 is an example of an eye camera connector, at 660 is an example of an EEG sensor connector and ADC module, at 670 is an example of a Wi-Fi module, and at 68 is an example of a processor module (e.g., Raspberry Pi Compute Module 3).

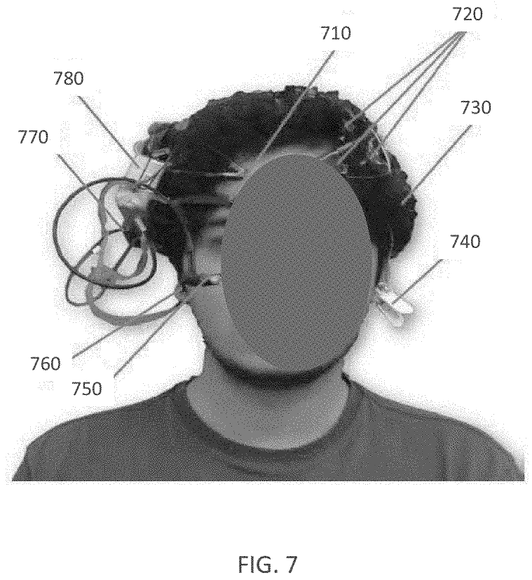

To evaluate the efficacy of the disclosed integrated headset (see FIG. 7), the components can be evaluated. Below are the evaluation results for each of the components.

The earlobe PPG module can be evaluated during rest and in active conditions. Heart rate can be measured while users are sitting and/or walking in place. The PPG sensor may be placed at the earlobe as in FIG. 7 and the changes in blood volume measured at the sampling rate of 100 Hz (or another frequency). At the same time or nearly the same time, the baseline can be collected using an EEG/ECG acquisition system sampled at 1 KHz. Three electrodes may be placed on the users' chest such that they are located over the heart, and on either side of the ribs. FIG. 7. depicts an example of a system including the integrated headset with world camera 710, EEG sensors 720, battery 730, EEG reference electrode 740, eye camera 750, earlobe PPG sensor 760, headphone/speaker connector 770, and controller 780.

In experiments, participants were sitting and/or walking, during which their ECG and PPG data were simultaneously measured. In each trial, two minutes of data were collected. For the walking condition, the participants were instructed to walk-in-place at a regular rate and ANC was performed to remove motion noise. Peak detection was used to find the heart beats in both signals for counting the heart rate.

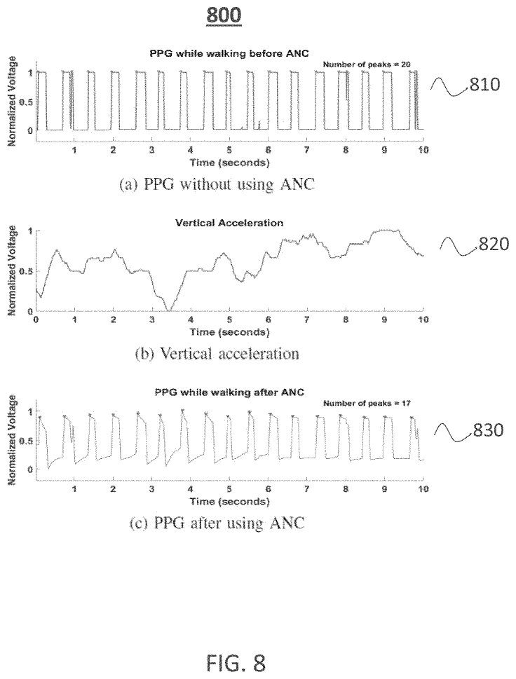

FIG. 8 depicts a comparison of 10-second waveforms from the earlobe PPG sensor before and after ANC using the vertical acceleration signal as a noise reference during walking. FIG. 8 shows the working of the 10th order ANC filter utilized on a 10-second interval of PPG data while walking. The PPG data at 810 is clipped at the top because a third order high-gain amplifier and band-pass filter were used thus amplifying the signal and making it easier to distinguish the peaks in PPG. From 810 the number of peaks in the waveform is computed to be 20, which is incorrect as the waveform is distorted. We then use the measure of the noise from vertical acceleration at 820 for the ANC filter. FIG. 8 at 830 shows the output of PPG data after using the ANC filter and as expected the erroneous peaks are eliminated, giving the total number of peaks as 17.

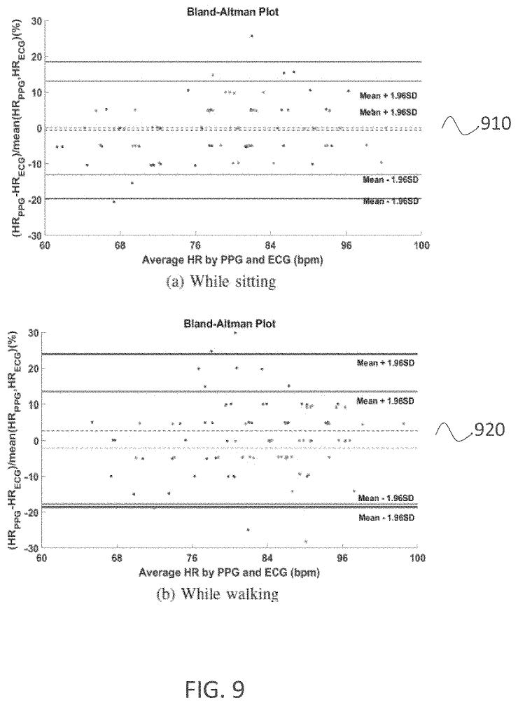

Bland-Altman analysis which is a general and effective statistical method for assessing the agreement between two clinical measurements was then performed to compare the heart rate computed by our PPG module to the true heart rate computed using the high-resolution ECG signal. Fifteen-second trials were used to calculate the HR using the peak detection. FIG. 9 at 910 shows the result of Bland-Altman analysis while the users were sitting. As can be seen, most of the trials are between the Mean 1.96 SD agreement threshold for both, with and without using ANC. Further, we see that using ANC decreases the agreement threshold, making the two signals more conforming. Similar results were obtained for the trials when users were walking (FIG. 9 at 920). Again using ANC makes the HR measures from the two signals more agreeable. Furthermore, for both cases, the trials from the two signals were almost always in agreement, indicating that our earlobe PPG module is capable of measuring heart rate with high accuracy.

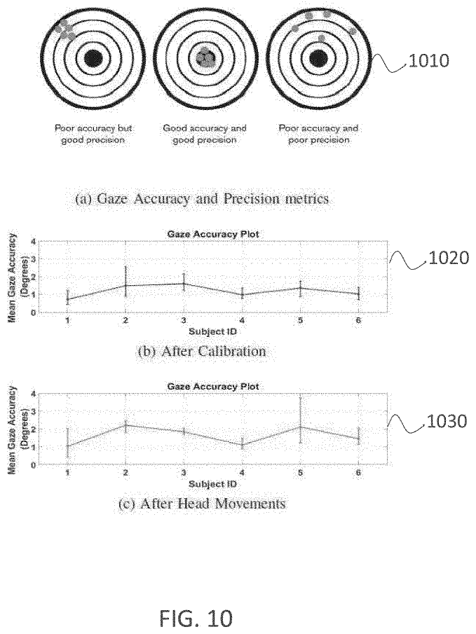

The performance of the paired eye pupil monitoring and world-view cameras in measuring eye-gaze were evaluated using a structured visual task to measure precision and accuracy during use. Gaze accuracy and precision was measured (FIG. 10 at 1010) for users following calibration (ideal setting) and after head movements (real-world use). In this task, the users were asked to calibrate their eye gaze using nine targets which appeared on a screen 2.5 feet away from them (such that >90% of the camera's field of view was composed of the task-screen.) For six subjects, a series of 20 targets were used randomly distributed on the screen to account for the majority of their field of view. This indicates the accuracy and precision measurements just after calibration. The participants were asked to move their head naturally for 30 seconds without removing the headset. This action was designed to simulate the active head-movement scenarios when wearing the headset because the gaze performance may not be reported after the subject has moved from his/her position. Similar to the above task, the subjects were asked to again focus on 20 different points appearing on the screen to assess the gaze performance after head movements. The foregoing process was repeated three times for each subject. No chin rest was used during or after calibration so that gaze performance is measured with natural head and body movements.

The accuracy is measured as the average angular offset—distance in degrees of the visual angle—between fixation locations and the corresponding fixation targets. The gaze accuracy obtained before and after head movements is shown in FIGS. 10 at 1010 and 1020. In the example implementation, the mean gaze accuracy over all the trials was found to be 1.21 degrees without and 1.63 degrees after head movements. Other implementations consistent with this disclosure may have higher or lower accuracy. The decrease in gaze accuracy after head movements is expected because the headset's position is displaced by a small value. For all the subjects, the mean gaze accuracy was mostly less than 2 degrees and the mean performance drifts only 0.42 degree, which is significantly less than 1-2-degree drift in commercially available eye-gaze systems.

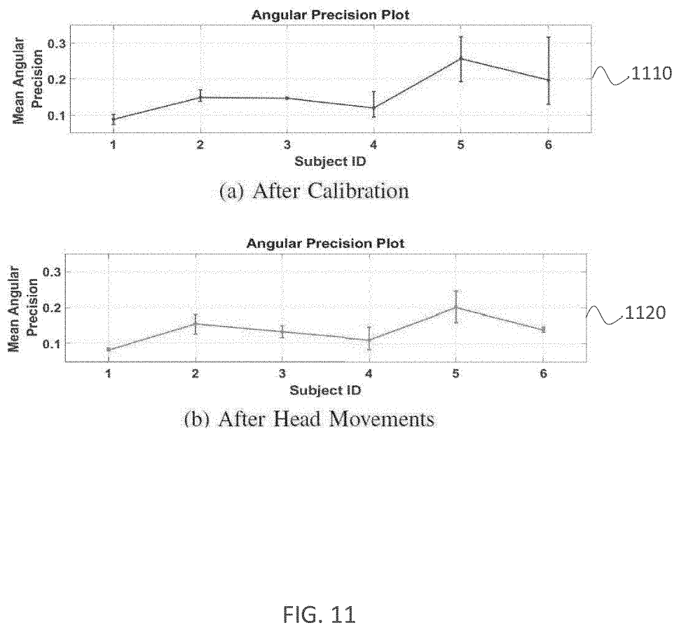

The precision may be measured as the root-mean-square of the angular distance between successive samples during a fixation. FIG. 11 shows example results of the angular precision for all the subjects. In the example implementation, the mean angular precision was found to be 0.16 and 0.14 before and after head movements. As clear from the figure, the degree of visual angle almost always within the range of 0.15. Furthermore, the precision has a mean shift post head movement of only 0.2, indicating a minimal angular distance shift comparable to existing systems.

The disclosed EEG sensors were compared to state-of-the-art dry EEG sensors by (e.g., Cognionics) to evaluate the signal correlation achieved using the two types of sensors. This comparison also demonstrates that the disclosed EEG sensors are actually acquiring EEG as opposed to just electromagnetic noise, and they are shielded from ambient noise in the real-world environment. The sensors were also evaluated on a steady-state visually evoked potentials (SSVEP) brain-computer interface (BCI) task to measure the sensors' performance measuring various frequencies during use.

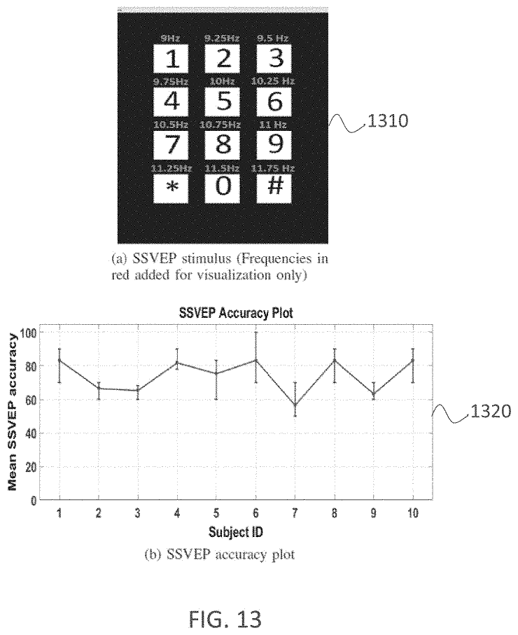

For the SSVEP testing, EEG sensors were placed at T5, O1, Oz, O2, and T6 sites according to the EEG 10-20 system. The location on and near the occipital lobe was chosen to evaluate the performance of our sensors because the SSVEPs response to repetitive visual stimuli of different frequencies is strongest over the occipital lobe. Ten subjects participated in this experiment constituting three trials of ten random numbers each to be typed using an SSVEP-based keypad on a mobile tablet (e.g., Samsung Galaxy S2) with an EEG sampling rate of 500 Hz (see FIG. 13 at 1310). The frequencies of the 12 stimuli on keypad varied between 9-11.75 Hz with increments of 0.25 Hz. This fine resolution increment was chosen to analyze the capability of sensors in distinguishing between minimally varying frequencies. The stimulus presentation time was 4 seconds with an interval of 1 second of blank screen between two consecutive stimuli. Only the middle 2 seconds was used from data from each stimulus for SSVEP analysis. To compare the signal quality obtained from the two types of sensors, a Cognionics sleep headband was to acquire EEG from one Cognionics sensor at the temporal lobe and one of our sensors next to it in a subset of subjects. The location was chosen so that hairs on the scalp are present around the sensors.

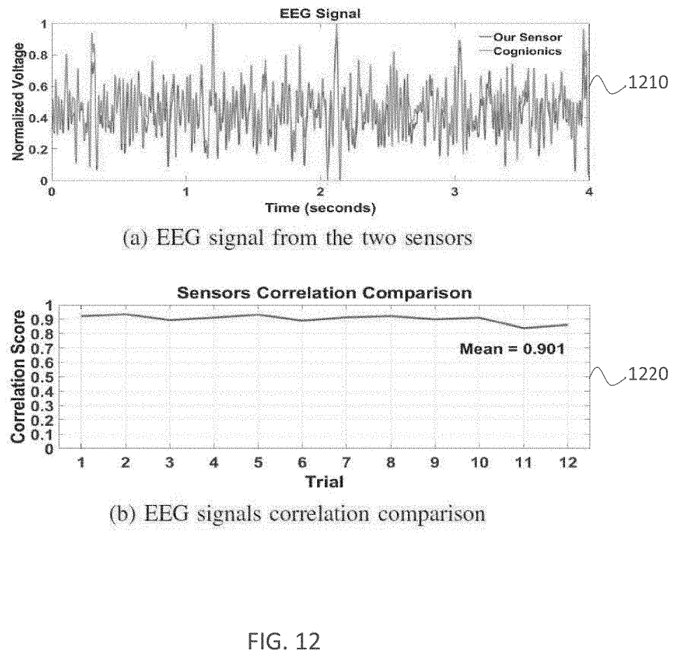

FIG. 12 at 1210 plots 4-seconds of EEG data acquired by the two sensors where a high correlation between the two signals is evident. FIG. 12 at 1220 plots the correlation of a subset of 12 of the trials. The correlation between the EEG signals acquired by the two different sensors is very high indicated by the mean correlation reaching 0.901, indicating the dry EEG sensor disclosed in the patent document are capable of measuring EEG signals from the hair-covered scalp areas.

FIG. 13 at 1310 shows the SSVEP keypad used as the visual stimulation of the BCI speller, and FIG. 13 at 1320 shows an example of an accuracy plot. As mentioned above, each subject needed to ‘type’ ten digits in each of the three trials. We computed the SSVEP classification performance using the filter-bank correlation analysis. This method does not require any training and is capable of working in real- time. As mentioned above, only the middle 2 seconds of EEG data during the 4-second stimulus presentation was used for evaluation. For almost all the subjects, the performance of SSVEP accuracy was very good (80% accuracy). There are some expected variations because it is well known that the signal-to-noise ratio of SSVEPs varies among individuals. The mean performance across all the subjects was 74.23%.

Bio-sensing technology is advancing rapidly both as a clinical research tool and applications in real-world settings. The existing bio-sensing systems are numerous and capable of measuring various physiological metrics in well-controlled laboratories. But, they are not practical for routine use by users in unconstrained real-world environments. Furthermore, they lack a method to automatically tag cognitively meaningful events. Repeatedly, it has been shown that using multiple bio-sensing modalities improves performance and robustness of decoding brain states and responses to cognitively meaningful real-life events. Hence, developing a research-grade wearable multi-modal bio-sensing system would allow us to study a wide range of previously unexplored research problems in real-world settings. Furthermore, because of the modular nature of our system, it is also capable of working with other individual sensing systems currently available to add modalities as required in the experimental setups.

While this patent document contains many specifics, these should not be construed as limitations on the scope of any invention or of what may be claimed, but rather as descriptions of features that may be specific to particular embodiments of particular inventions. Certain features that are described in this patent document in the context of separate embodiments can also be implemented in combination in a single embodiment. Conversely, various features that are described in the context of a single embodiment can also be implemented in multiple embodiments separately or in any suitable subcombination. Moreover, although features may be described above as acting in certain combinations and even initially claimed as such, one or more features from a claimed combination can in some cases be excised from the combination, and the claimed combination may be directed to a subcombination or variation of a subcombination. Although some specific components are listed in the foregoing, other components may be used in place of, or in addition to, those listed.

Similarly, while operations are depicted in the drawings in a particular order, this should not be understood as requiring that such operations be performed in the particular order shown or in sequential order, or that all illustrated operations be performed, to achieve desirable results. Moreover, the separation of various system components in the embodiments described in this patent document should not be understood as requiring such separation in all embodiments.

Only a few implementations and examples are described and other implementations, enhancements, and variations can be made based on what is described and illustrated in this patent document.

1. A multi-modal bio-sensing apparatus, comprising:
a first sensor module comprising a photoplethysmogram (PPG) sensor configured to produce a first output representative of a blood volume of a user, wherein the PPG sensor is configured to remove from the first output an error signal due to movement of the user;
a second sensor module comprising an electroencephalogram (EEG) sensor configured to produce a third output representative of brain neural activity of the user;
a third sensor module comprising an eye-gaze camera configured to capture a gaze direction of one or more eyes of the user; and
a wireless communications transceiver coupled to receive sensor data from the first sensor module, the second sensor module, or the third sensor module and configured to wirelessly transmit the received sensor data from the first sensor module, the second sensor module, or the third sensor module out of the multi-modal bio-sensing apparatus.

2. The multi-modal bio-sensing apparatus of claim 1, wherein the error signal is determined from a second output from an accelerometer attached to the compact multi-modal bio-sensing apparatus, and wherein the error signal is removed from the first output using an adaptive filter.

3. The multi-modal bio-sensing apparatus of claim 1, further comprising:
one or more galvanic skin response (GSR) sensors configured to determine an impedance of the skin of the individual.

4. The multi-modal bio-sensing apparatus of claim 1, further comprising:
a worldview camera configured to capture a scene around the compact multi-modal bio-sensing apparatus.

5. The multi-modal bio-sensing apparatus of claim 1, further comprising:
a battery power source to provide power to the first sensor module, the second sensor module, the third sensor module, and the wireless communications receiver, wherein the compact multi-modal bio-sensing apparatus is mobile with freedom for the user to move about.

6. The multi-modal bio-sensing apparatus of claim 1, further comprising:
a headphone or speaker;
at least one processor and at least one memory containing executable instructions to cause the data to be sent to another transceiver; and
at least another memory configured to store the data prior to transmission.

7. The multi-modal bio-sensing apparatus of claim 1, wherein the eye-gaze camera comprises an infrared camera.

8. The multi-modal bio-sensing apparatus of claim 1, wherein the EEG sensor comprises:
a plurality of electrode sensors, each electrode sensor structured to include an electrode tip that is electrically conductive and an electrically conductive cage formed to enclose the electrode tip to form a Faraday cage to shield the electrode tip from external electromagnetic interference; and
an EEG control module coupled to the electrode sensors to apply and receive electrical signals from the electrode sensors.

9. The multi-modal bio-sensing apparatus of claim of claim 8, wherein the electrode tip comprises silver and epoxy, and the Faraday cage is formed by an electrically conductive tape.

10. The multi-modal bio-sensing apparatus of claim of claim 9, wherein the electrically conductive tape comprises copper (Cu).

11. The multi-modal bio-sensing apparatus of claim 8, wherein each electrode sensor includes an amplifier circuit coupled to the electrode tip to provide electrical signal amplification, and the wherein the amplifier circuit is enclosed by the Faraday cage.

12. The multi-modal bio-sensing apparatus of claim 4, wherein one or more objects captured on video from the worldview camera are identified in the gaze direction by computer vision, and an associated time-stamp recorded to indicate one or more event times around which sensor data is recorded.

13. The multi-modal bio-sensing apparatus of claim 12, wherein the computer vision is trained on one or more classes of objects.

14. The multi-modal bio-sensing apparatus of claim 1, wherein every data point of at least the PPG sensor, the EEG sensor, and the eye gaze camera is time-stamped for data synchronization.

15. A multi-modal bio-sensing method, comprising:
sensing, by a photoplethysmogram (PPG) sensor, a blood volume of a user and generating an output representative of the blood volume;
removing, from the output, an error signal due to a movement of the user;
sensing, by an electroencephalogram (EEG) sensor, brain neural activity of the user;
determining, by an eye-gaze camera, a gaze direction of one or more eyes of the user; and
transmitting, by a wireless transceiver, one or more of data representative of the blood volume with the error signal removed, data representative of brain neural activity, or the gaze direction of the user.

16. The multi-modal bio-sensing method of claim 15, wherein the error signal is determined from an accelerometer, and wherein the error signal is removed from the output using an adaptive filter.

17. The multi-modal bio-sensing method of claim 15, further comprising:
sensing, by one or more galvanic skin response (GSR) sensors, an impedance of the skin of the user.

18. The multi-modal bio-sensing method of claim 15, further comprising:
capturing, by a worldview camera, a scene in an area around one or more of the PPG sensor, the EEG sensor, or the eye-gaze camera.

19. The multi-modal bio-sensing method of claim 15, further comprising:
powering, by a battery power source, one or more of the PPG sensor, the EEG sensor, or the eye-gaze camera, and the wireless transceiver.

20. The multi-modal bio-sensing method of claim 15, wherein the eye-gaze camera comprises an infrared camera.

21. The multi-modal bio-sensing method of claim 15, wherein the EEG sensor comprises:
a plurality of electrode sensors, each electrode sensor structured to include an electrode tip that is electrically conductive and an electrically conductive cage formed to enclose the electrode tip to form a Faraday cage to shield the electrode tip from external electromagnetic interference; and
an EEG control module coupled to the electrode sensors to apply and receive electrical signals from the electrode sensors.

22. The multi-modal bio-sensing method of claim 21, wherein
the electrode tip comprises silver and epoxy, and wherein the Faraday cage is formed by an electrically conductive tape.

23. The multi-modal bio-sensing method of claim 22, wherein the electrically conductive tape includes copper (Cu).

24. The multi-modal bio-sensing method of claim 21, wherein each electrode sensor includes an amplifier circuit coupled to the electrode tip to provide electrical signal amplification, and wherein the amplifier circuit is enclosed by the Faraday cage.

25. The multi-modal bio-sensing method of claim 18, wherein one or more objects captured on video from the worldview camera are identified in the gaze direction by computer vision, and an associated time-stamp recorded to indicate one or more event times around which sensor data is recorded.

26. The multi-modal bio-sensing method of claim 18, wherein the computer vision is trained on one or more classes of objects.

27. The multi-modal bio-sensing method of claim 15, wherein every data point of at least the PPG sensor, the EEG sensor, and the eye gaze camera is time-stamped for data synchronization.
